(19) United States
(12) Patent Application Publication
(10) Pub. No.: US 2003/0008003 A1
(43) Pub. Date: Jan. 9, 2003

(54) Compositions for enhanced absorption of NSAIDs

(76) Inventor: Fahkreddin Jamali, Edmonton (CA)

Correspondence Address: CAHN & SAMUELS LLP, 2000 P STREET NW, SUITE 200, WASHINGTON, DC, 20036, US

(21) Appl. No.: 10/119,313

(22) Filed: Apr. 10, 2002

Related U.S. Application Data
(60) Provisional application No. 60/282,497, filed on Apr. 10, 2001.

(51) Int. Cl. A61K 31/405; A61K 9/20; A61K 31/192

(52) U.S. Cl. 424/465; 514/420; 514/570

(57) ABSTRACT

The invention is a composition and method for treating acute pain using a composition containing one or more NSAIDs. The preferred composition includes ibuprofen, sodium bicarbonate, and Gelucire.

### CROSS-REFERENCE TO RELATED APPLICATIONS

Not applicable.

### STATEMENT REGARDING FEDERALLY SPONSORED RESEARCH

Not applicable.

### BACKGROUND OF THE INVENTION

1. Field of the Invention

The present invention is directed to NSAID formulations having increased absorption in suppressed vagal systems. One of the primary NSAIDs, (±)-2-(4-Isobutylphenyl)propionic acid, ibuprofen, is a potent and well tolerated anti-inflammatory, analgesic, and anti-pyretic compound.

2. Description of Related Art

In the treatment of acute pain rapid absorption of orally administered analgesics is desirable. For non-steroidal anti-inflammatory drugs (NSAIDs), such as ibuprofen and ketoprofen, there appears to be a positive relationship between plasma drug concentration and analgesic activity. Any delay in absorption or reduction in the circulating drug concentration may result in treatment failure or in reduced activity of the analgesic. One skilled in the art readily recognizes that analgesic formulations with enhanced absorption rates are expected to be more effective in treating acute pain.

However, none of the widely available solid dosage forms of NSAIDs have been claimed to be superior over the products of the same drug with respect to onset of action. This is despite differences in apparent rate of absorption usually measured in healthy volunteers. It appears that rapid absorption observed in healthy subjects does not necessarily result in a quick onset of action in patients experiencing pain.

Jamali & Kunz, Brit J. Clin. Pharmacol., 47:391-396 (1999) have reported that, using dental surgery as a marker of pain, pain or its associated trauma causes reduced rate of absorption of ibuprofen. The publication details the absorption rates for two doses of ibuprofen, 200 mg and 600 mg. Surgery resulted in a two hour delay in the mean time to peak concentration, significant decreases in serum ibuprofen concentrations following both doses, and a fall to sub-optimal serum concentrations following the 200 mg dose.

For example, during the first two hours after the 200 mg dose, dental extraction resulted in a significant reduction of the area under serum drug concentration (AUC _{0-2h,} mg/L⁻¹/h) from 5.6±2.9 to 1.6±1.8 (p<0.01) and from 5.5±3.0 to 2.1±2.0 (p<0.05) for S and R-ibuprofen, respectively. Similar observations were made following the 600 mg dose for AUC₀₋₂ₕ of S-ibuprofen (from 14.2±6.1 to 7.2±5.5 mg. L⁻¹.h, p<0.05) with no significant difference for R-ibuprofen (form 14.4±9.5 to 5.8±7.1). AUC₀₋₆ₕ was also significantly reduced by surgery.

The publication concludes that wisdom tooth removal, as an example of a person in pain, resulted in substantial decreases in the serum concentration of ibuprofen enantiomers and an increase in the period to peak concentration.

Thus, dental patients may experience a delayed response and possible treatment failure when taking ibuprofen for pain relief after surgery.

The observed reduced absorption is believed to be caused by suppression of the vagal nervous system. The vagus nerve, nervus vagus, is the 10th cranial nerve; suppressing the activity of the vagus nerve causes reduced gastric juice secretion and motility, both of which are associated with decreased absorption of NSAIDs. Sufficient fluid and a rather quick exit from stomach (hence entry to small intestine, the major site of absorption) is needed for efficient absorption.

In another indicia of the inventor's belief that the bioavailability of a composition for an animal in pain is different than the same composition in an animal not in pain, it is now known that for some NSAIDs for which there are active and non-active isomers, e.g., ibuprofen, the conversion of the non-active isomer to the active isomer occurs predominately only when the animal is not in pain. For example, it has now been shown that the (R) isomer of ibuprofen (non-active) does not as readily convert to the (S) isomer (active) when the animal/human is in pain; the lack of conversion to the (S) isomer correlates with decreased absorption and decreased pain relief.

The problem of decreased absorption in vagally suppressed mammals is further exacerbated by the relative insolubility of NSAIDs in an aqueous or gastric (acidic) environment. Finally, there is growing evidence that these conditions, namely, reduction in stomach motility, stomach secretion diminution, and reduced absorption appear to be present in the elderly, or what shall be termed herein, the geriatric stomach.

Some prior art formulations, such as U.S. Pat. Nos. 6,197,336 and 4,834,966, dissolve the ibuprofen formulation prior to administering the composition.

Other prior art formulations, e.g., PCT/EP97/00841, incorporate an alkali metal bicarbonate into the ibuprofen formulation to enhance the compressibility of the solid dosage form. These formulations include ibuprofen as the active agent, the bicarbonate as a compressibility enhancer, a compressible filler, and a disintegrant (preferably croscarmellose sodium or sodium starch glycollate).

Alkali metal carbonates and bicarbonates are soluble materials which have previously been proposed for use in effervescent tablets, for example to react with the acid component in an effervescent couple (see for example WO 94/10994) or to prevent initiation of the effervescent reaction e.g. during storage.

Effervescent tablets disintegrate by means of the reaction between acid and base particularly in the presence of water leading to the production of carbon dioxide. The system of disintegration of non-effervescent dosage forms according to the present invention, which are arranged to be swallowed and for which an effervescent reaction is not desired, is different to that of effervescent systems. The present dosage form does not contain any soluble acidic component with which the alkali metal carbonate or bicarbonate could react in an effervescent reaction.

### SUMMARY OF THE INVENTION

It is desirable to provide an NSAID formulation that can deliver drugs into the blood stream despite a suppressed vagal system.

It would be advantageous to provide a composition having enhanced absorption of NSAIDs, which tend to be poorly water soluble, as well as providing an improved concentration of the drug at the cellular level at the site of its action. It would also be advantageous to provide a method and composition for increasing the absorption rate of such poorly water-soluble active agents by increasing the disintegration efficiency of the composition in tablet form, by accelerating the time and speed of the tablet disintegrating into molecules in solution, and by increasing the speed by which active agent is available in solution for absorption.

NSAIDs (or aspirin-like drugs) are typically categorized into six structural groups. The first group are the salicylic acids and esters, including but not limited to, aspirin, benorylate, aloxiprin, salsalate and choline magnesium trisalicylate. The second are the propionic acid derivatives, including, but not limited to, ibuprofen, naproxen, flurbiprofen, ketoprofen, fenoprofen, fenbufen, benoxaprofen and suprofen. The third is the class of oxicams, including, but not limited to, piroxicam and meloxicam. The fourth are acetic acid derivatives, such as phenylacetic acids, including but not limited to, diclofenac, ketorolac, and fenclofenac; and carbo- and heterocyclic acetic acids, including but not limited to, indoles such as indomethacin and sulindac, and pyrroles, such as tolmetin. The fifth are the pyrazolones, including but not limited to, oxyphenbutazone, phenylbutazone, feprazone and azapropazone. The sixth are the fenamic acid derivatives, including but not limited to, flufenamic acid and mefenamic acid.

Ibuprofen is sold under the trade mark BRUFEN (Boots Company). Other trade marks in the UK for ibuprofen are FENBID and APSIFEN, and in the U.S. are RUFEN, ADVIL, MOTRIN and NUPRIN. Ibuprofen is poorly soluble in water: less than 1 part of drug will dissolve in 10,000 parts of water. However, it is fairly soluble in simple organic solvents. The most frequent adverse effect reported is gastrointestinal. The drug is well absorbed and extensively bound to plasma proteins in vivo. It is prescribed for rheumatic arthritis and other musculoskeletal disorders, as well as acute gout. The dosage of the drug is typically from 600 to 1200 mg daily in divided doses, with 2,400 mg per day being the maximum.

Ibuprofen is also indicated for use in the treatment of rheumatoid arthritis, osteoarthritis, ankylosing spondylitis, seronegative arthropathies, periarticular disorders and soft tissue injuries. Ibuprofen may also be used in the treatment of postoperative pain, postpartum pain, dental pain, dysmenorrhoea, headache, musculoskeletal pain or the pain or discomfort associated with the following:

respiratory infections, colds or influenza, gout or morning stiffness. A critical factor relating to the use of ibuprofen to treat the above disorders concerns, as noted above, improving the onset of action of ibuprofen, particularly in the treatment of pain. This issue partially concerns improving the amount and speed of achieving a certain blood serum level of ibuprofen. It is believed that rapid disintegration of a formulation, beginning in the mouth, but primarily in the stomach, releases the drug into the body more quickly, thereby leading to a more rapid onset of therapeutic action, as compared with a standard dosage form or with dosage forms calibrated against healthy individuals. Accordingly, it is desired to produce a solid dosage form for oral administration adapted to disintegrate quickly in the gastrointestinal tract. It is also preferred that the dosage form is manufactured by compression on standard tableting machines.

(±)-2-(4-Isobutylphenyl)propionic acid, ibuprofen, is a potent and well tolerated anti-inflammatory, analgesic and anti-pyretic compound. The racemic drug consists of equal parts of two enantiomers, namely S(+)-2-(4-isobutylphenyl)propionic acid or S(+)-ibuprofen and R(−)-2-(4-isobutylphenyl)propionic acid or R(−)-ibuprofen. It is known that S(+)-ibuprofen is the active agent and that R(−)-ibuprofen is partially converted into S(+)-ibuprofen in humans.

In accordance with one embodiment of the present invention, the composition contains an NSAID, preferably ibuprofen (hereinafter referred to as IB); a disintegration and dissolution agent, such as a bicarbonate, preferably sodium bicarbonate; and an ester of a fatty acid as an anti-precipitation agent.

These ingredients are formed into a tablet or solid form, a tablet having enhanced disintegration into particles and subsequently enhanced dissolution of the particles into dispersed molecules in solution.

In accordance with the present invention, the bicarbonate is a disintegrator or disintegrating agent that increases the solubility of the NSAID. The anti-precipitant provides an interface between lipid and aqueous phases (i.e., under gastric conditions) and prevents and/or reduces precipitation of the ibuprofen in the gastric environment. While not intending to be limited to a particular mechanism of action, the inventor believes that the bicarbonate increases solubility by promoting the formation of sodium ibuprofen, a salt that is readily converted to ibuprofen; ibuprofen precipitates under gastric conditions, so the anti-precipitation agent prevents precipitation by increasing the solubility of the ibuprofen in the gastric environment.

For example, the sodium salt of ibuprofen may precipitate out in an acidic environment such as the stomach, thus reducing the amount of active ingredient available for absorption. The inclusion of anti-precipitants, such as gelucire and other similar compounds, may be desirable in a composition of the present invention in order to prevent or reduce the amount of active ingredient that precipitates in an acidic environment.

The compositions and methods of the present invention achieve chemically what happens biologically when NSAIDS are administered and absorbed in healthy subjects. Biologically, the stomach has a certain amount of movement or motility, as well as gastric juice, that contribute to a tablet disintegrating into particles, and then dissolving into molecules.

In a vagally suppressed human, i.e., a human in pain and/or the geriatric stomach, both the motility and gastric juice extraction are reduced. This results in delayed absorption. The present invention accelerates the time line of disintegration into particle form by chemically mimicking the agitation provided by the motility function, by initiating the disintegration from tablet form into particles as soon as the tablet is exposed to a very limited amount of fluid. In the presence of some moisture, the incorporated bicarbonate starts reacting with ibuprofen. The result is breaking down of the larger solid particles, enhancing solubility, and providing a greater amount of active agent earlier in the process, thereby accelerating the absorption rate, and thereby providing more relief, faster.

The compositions and methods of the present invention achieve this result by surrounding, capturing, or formulating active agent particles, such as ibuprofen, in a matrix or the like of a disintegrating agent that, upon exposure to an aqueous environment, promotes the break-up of the tablet into smaller particles of active agent, thereby increasing the availability of the active agent for absorption.

The solid dosage forms according to the invention are adapted for direct administration to a patient to obtain the desired therapeutic effect. They are not intended to be dissolved or dispersed in water prior to administration.

Furthermore, the compressed dosage forms according to the present invention need no further processing after compression of a composition comprising a mixture of the ingredients to produce a solid dosage form.

The accompanying drawings show illustrative embodiments of the invention from which these and other of the objectives, novel features and advantages will be readily apparent.

### DESCRIPTION OF THE DRAWINGS

FIG. 1 shows plasma ibuprofen concentration in a representative patient a week before (i.e., healthy) and just after (i.e., in pain) dental extraction. FIG. 1 is used to show that the serum level of ibuprofen in healthy patients does not correlate to the serum level of ibuprofen in patients who are in pain.

FIG. 2 graphically shows the suitability of an animal model (Is this the first time you are referring to the animal model?) used as an indicator of a human response, and used to test various formulations of the present invention.

FIG. 3 shows the plasma concentration time curve after the oral administration of a composition of the present invention (Formulation 1) versus Motrin (n=6 per group).

FIG. 4 shows the plasma concentration time curve after the oral administration of a composition of the present invention (Formulation 2) versus Motrin (n=6 per group).

FIG. 5 shows the plasma concentration time curve after the oral administration of a composition of the present invention (Formulation 3; n=6) versus Motrin (n=5, per group).

FIG. 6 shows the enhanced absorption characteristics of the compositions of the present invention as compared to Motrin. What is this one?

FIG. 7 shows the comparative dissolution profiles among ibuprofen alone; ibuprofen and sodium bicarbonate; and ibuprofen, sodium bicarbonate, and gelucire.

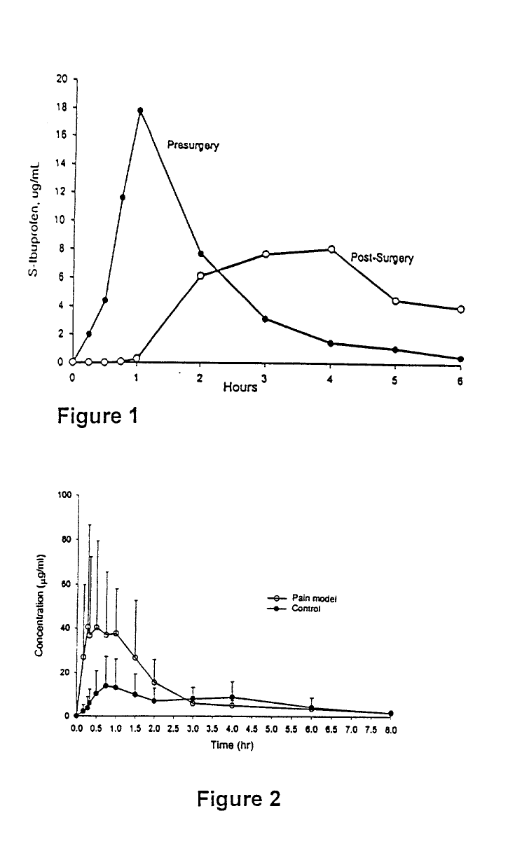

### DETAILED DESCRIPTION OF THE INVENTION

The present invention is a composition containing an NSAID as an active agent, said composition having increased absorption in vagally suppressed systems. The composition may comprise an NSAID and a disintegration and dissolution agent, such as a bicarbonate. The composition may further include an anti-precipitation agent.

The present invention is also a composition comprising ibuprofen, and a disintegration and dissolution agent, such as a bicarbonate. The invention also includes a method of treating inflammation or alleviating pain comprising administering a composition as described in this paragraph.

The present invention is also a composition comprising ibuprofen, a disintegration and dissolution agent, such as a bicarbonate, and an anti-precipitation agent. The preferred anti-precipitation agent is Gelucire. Such a composition is characterized by having increased absorption of the active agent, as compared to other compositions when the comparison assesses the absorption of the active agent under pain conditions. The invention also includes a method of treating inflammation or alleviating pain comprising administering a composition as described in this paragraph.

The present invention is also any of the above compositions, further comprising one or more lubricating agents, one or ore binders, one or more additional disintegrating agents, one or more flow aids, and/or one or more colorants and/or flavorants.

The present invention is also a method for increasing the absorption of an NSAID-containing composition, said method comprising providing a composition, such as one of the compositions described above, whose ingredients are specifically formulated to increase absorption under pain conditions, i.e., in a vagally suppressed system.

The present invention is also a method of treating acute pain in humans comprising administering a composition according to the present invention.

It will be appreciated that the present invention provides a method of treating inflammation, pain and pyrexia by administration of a pharmaceutical composition comprising racemic ibuprofen, together with a pharmaceutically acceptable carrier to a mammal, e.g. a human, in need thereof. Preferably the ibuprofen is present in one or more of its well known forms, namely, ibuprofen, its S(+) and R(−) enantiomers, including different enantiomeric ratios thereof, salts, hydrates, and other derivatives. The preferred form is a dihydrate. The most preferred form is the acid form.

The ibuprofen may be also present in the form of any salt or other derivative of ibuprofen or its enantiomers. If necessary, the ibuprofen may comprise one or more ibuprofen active ingredients such as racemic ibuprofen and S(+)-ibuprofen in combination. However, we prefer that the ibuprofen comprises a single ibuprofen active ingredient. The ibuprofen active agent may also be present in different degrees of hydration. The present invention applies to both anhydrous and hydrated forms, for example the monohydrate or the dihydrate. The most stable anhydrous or hydrated form will generally be used. Preferably, the ibuprofen is in the form of a salt of racemic or S(+)-ibuprofen. Representative examples include alkali metal salts, for example the sodium or potassium salts of ibuprofen; alkaline earth metal salts, e.g. the calcium or magnesium salts of ibuprofen; metal salts, e.g. the aluminium salt of ibuprofen; amino acid salts for example the lysine or arginine salts of ibuprofen: or amine salts, e.g. the meglumine salt of ibuprofen. Preferably the ibuprofen is a single salt selected from alkali metal salts, amino acid salts and amine salts. These soluble ibuprofen salts also have the advantage that, as they are more soluble in an aqueous medium, on release from the formulation they have improved absorption, thus leading to an improved onset of action compared to the substantially insoluble forms of ibuprofen. The sodium salt of ibuprofen is particularly preferred, especially the sodium salt of racemic ibuprofen. It has been found that the dihydrate of the sodium salt of racemic ibuprofen is a particularly stable hydrated form, accordingly we prefer to use the sodium salt dihydrate in a compressed dosage form according to the present invention.

The compositions and methods of the present invention are particularly suited to forming non-aqueous granulations and to solid non-effervescent dosage forms.

The present invention further relates to the use of the above composition to provide tablets and granules that are fast dissolving and fast acting. The granulation and tableting composition also includes normal excipients useful for the preparation of tablets.

The present invention is also a composition comprising an NSAID as an active agent, and a bicarbonate as a disintegrating agent. The composition may further comprise one or more of the following: one or more diluents or fillers; one or more binders or adhesives; one or more additional disintegrating agents; one or more lubricating agents; and one or more miscellaneous adjuncts, such as colorants and/or flavorants, any of said adjuncts being well known to those skilled in the art. Any number of pharmaceutically active agents may be employed in the formulations of the present invention. These active agents may exist as either solids or liquids at standard temperature and pressure. Exemplary pharmaceutically active agents suitable for use herein include, but are not limited to, the non-steroidal anti-inflammatory agents such as piroxicam, indomethacin, fenoprofen, meloxicam, and ibuprofen. In a preferred embodiment of the invention, the composition and method includes ibuprofen as the active agent.

The compositions of the invention may contain about 1-99% by weight of an NSAID, such as ibuprofen, preferably up to about 60% by weight, more preferably from about 15% to about 50% by weight; and 10-60% by weight of a bicarbonate, preferably between about 20% and 50%, and more preferably, between about 30% and 40%, And, in compositions that include an anti-precipitant, preferably up to about 5% by weight, more preferably from about 1% to about 30% by weight, and most preferably, from about 5% to about 7% by weight.

The compositions of the invention are generally prepared in unit dosage form. Preferably the unit dosage of ibuprofen is in the range of 10-1200 mg in a pre-calculated amount to provide doses which are equivalent by weight to doses of for example 100 mg, 200 mg, 400 mg or 800 mg of ibuprofen.

The bicarbonate can be any bicarbonate salt that is pharmaceutically acceptable, preferably sodium or potassium bicarbonate. The alkali metal carbonate or bicarbonate used in accordance with the present invention may suitably comprise sodium carbonate or bicarbonate or potassium carbonate or bicarbonate either alone or mixed together. Preferably, the alkali metal comprises sodium, thus sodium bicarbonate and sodium bicarbonate are preferred ingredients. The alkali metal carbonates may be supplied anhydrous or in varying degrees of hydration for example the monohydrate and decahydrate. Any of these forms may be used.

In therapeutic use, ibuprofen may be administered orally, rectally, or topically, preferably orally or topically. Suitably the therapeutic compositions of the present invention may take the form of any of the known pharmaceutical compositions for oral, rectal, or topical administration. Pharmaceutically acceptable carriers suitable for use in such compositions are well known in the art of pharmacy.

Solid compositions for oral administration are preferred compositions of the invention and there are known pharmaceutical forms for such administration, for example tablets and capsules.

Within the context of the present description the identity of the components and amounts thereof refer to the weight and identity of the starting materials used in preparing the composition. It is possible that during preparation of the composition and/or tablets, some interaction or reaction may occur between two or more components. To the extent that such interaction or reaction occurs the present invention is intended to cover such occurrences.

Normal excipients useful in the preparation of the tablets include, but are not limited to: lubricants such as magnesium stearate, sodium stearyl fumarate and sodium benzoate; anti-adherents such as talc and polyethylenglycol; glidants such as colloidal silica; diluents such as dicalcium phosphate, cellulose (for example microcrystalline cellulose) and its derivatives, carbohydrates and polyalcohols such as saccharose, xylitol and lactose; disintegrants such as crosslinked vinylic polymers (such as crosslinked PVP), derivatives of starch and of cellulose such as sodium carboxymethyl-starch and sodium croscarmelose; wetting agents such as TWEEN 80 (Trademark registered by ICI of Americas for polysorbate) and sodium lauryl sulphate.

Suitable excipients and their amounts can be readily determined by those of ordinary skill in the art according to the methods normally used in pharmaceutical technology. However, in the present invention, it is important to avoid excipients that would cause a significant decrease in tablet dissolution rate. Further, excipients must allow a good workability of the tablet.

In preparing the tablet of the present invention it is preferable to prepare an IB granulate, to mix it with the bicarbonate and the excipients, and then to compress.

An exemplary solid composition according to the invention may include a) 1-99% ibuprofen (preferably 15-60%); b) 1-90% of a diluent preferably 40-85%) and c) 0.5-25% of a solubilizer (preferably 1-10%) 0.1-10% of a lubricating agent (preferably 0.5 to 5%), d) 1-50% of a disintegrating agent (preferably 2-20%) and optionally e) 0.1-15% of a binder. Optionally 0.1-10% of a flow aid may be added. It will be appreciated by those skilled in the art that a particular excipient may perform more than one function for example maize starch may act as a diluent, a binder or as a disintegrating agent.

A preferred process for preparing a solid composition in tablet form comprises combining 10-90% of ibuprofen with 1-90% of a diluent, optionally adding other pharmaceutically acceptable excipients selected from lubricating agents, disintegrating agents, binders, flow aids, oils, fats and waxes, mixing the ingredients with one another to form a uniform mixture, and compressing the mixture thus obtained to form tablets which may be optionally coated with a film coat or a sugar-coat. In a most preferred process for preparing a solid composition in tablet form, an active ingredient such as ibuprofen is mixed with a bicarbonate, such as sodium bicarbonate under non-aqueous conditions. For example, in a conventional granulation step, ibuprofen and sodium bicarbonate are combined using isopropyl alcohol as the diluent.

Preferably the diluent includes lactose, calcium phosphate, dextrin, microcrystalline cellulose, sucrose, starch, calcium sulphate, sodium bicarbonate, or mixtures thereof.

Preferably the lubricating agent includes magnesium stearate, stearic acid, calcium stearate, sodium bicarbonate, or mixtures thereof. More preferably the lubricating agent is magnesium stearate or stearic acid.

Preferably the disintegrating agent includes microcrystalline cellulose, maize starch, sodium starch glycollate, low substituted hydroxypropyl cellulose, alginic acid or croscarmellose sodium, sodium bicarbonate, or mixtures thereof.

Preferably the binder includes polyvinyl pyrrolidone, gelatin, gelucire, hydroxypropylmethyl cellulose, starch, or mixtures thereof.

Suitable flow aids include, but are not limited to talc and colloidal silicon dioxide.

Liquid fill compositions (for example, viscous liquid fills, liquid paste fills, or thixotropic liquid fills) are also suitable for oral administration. Melt filled compositions may be obtained by mixing ibuprofen with certain esters of natural vegetable oil fatty acids, for example, the Gelucire (Trademark) range available from Gattefosse to provide a variety of release rates. Suitably a melt-filled capsule comprises a) 10-80% ibuprofen and b) 20-90% of a fatty acid ester excipient which comprises one or more polyol esters and triglycerides of natural vegetable oil fatty acids.

Suitable pharmaceutically acceptable hydrophobic carriers include the glycerides and partial glycerides. The preferred carriers are known under the trademark Gelucire, and are commercially available from Gattefosse Corporation, Hawthorne, N.Y. Gelucires are available with varying physical characteristics such as melting point, HLB and solubilities in various solvents. The preferred Gelucire is Gelucire 44/14.

For example, a tablet of the present invention may include 1-99% of an ibuprofen acid; about 10 to about 60% by weight of a bicarbonate; and 20-90% of a fatty acid ester excipient which comprises one or more polyol esters and triglycerides of natural vegetable oil fatty acids. The use of esters of fatty acids, e.g., Gelucire, is well known to those skilled in the art, as is evident from the number of patents that disclose its use. Exemplary patents include, but are not limited to U.S. Pat. Nos. 6,361,796; 6,312,704; 6,251,426; 6,242,000, and 6,238,689, among many others.

The compositions of the present invention may additionally comprise a taste masking component for example a sweetener, a flavoring agent, arginine, sodium carbonate or sodium bicarbonate.

Solid non-effervescent compositions are preferred compositions of the present invention. The preferred compositions are preferably formed into a tablet.

In the compositions of the present invention the NSAID, such as ibuprofen, may, if desired, be associated with other compatible pharmacologically active ingredients and/or enhancing agents. Thus, for example, ibuprofen may be combined with any ingredient commonly used in a cough or cold remedy, for example, an antihistamine, caffeine or another xanthine derivative, a cough suppressant, a decongestant, an expectorant, a muscle relaxant, or combinations thereof. Exemplary compatible pharmacologically active ingredients include, but are not limited to codeine, oxycodone, hydrocodone, and/or hydromorphone.

Suitable antihistamines which are preferably non-sedating include acrivastine, astemizole, azatadine, azelastine, bromodiphenhyrdramine, brompheniramine, carbinoxamine, cetirizine, chlorpheniramine, cyproheptadine, dexbrompheniramine, dexchlorpheniramine, diphenhydramine, ebastine, ketotifen, lodoxamide, loratidine, levocubastine, mequitazine, oxatomide, phenindamine, phenyltoloxamine, pyrilamine, setastine, tazifylline, temelastine, terfenadine, tripelennamine or triprolidine. Suitable cough suppressants include caramiphen, codeine or dextromethorphan. Suitable decongestants include pseudoephedrine, phenylpropanolamine and phenylephrine. Suitable expectorants include guaifensin, potassium citrate, potassium guaiacolsulphonate, potassium sulphate and terpin hydrate.

In another aspect the present invention provides a method of preparing a pharmaceutical composition comprising IB together with sodium bicarbonate as an absorption aide. Ibuprofen and bicarbonate are administered in a solid dosage form which upon exposure to stomach juice they start to react to one another. This provides first disintegration, second, motion and third, increased solubility. The increased solubility is maintained by the presence of gelucire.

In a further aspect the present invention provides a process to prepare a pharmaceutical composition comprising IB and a disintegrating agent, together with a pharmaceutically acceptable carrier comprising combining IB in solid form with a pharmaceutically acceptable carrier and formulating into a dosage form.

A preferred process for preparing a solid composition in tablet form comprises combining 10-90% of IB with 1-90% of a diluent, optionally adding other pharmaceutically acceptable excipients selected from lubricating agents, disintegrating agents, binders, flow aids, oils, fats and waxes, mixing the ingredients with one another to form a uniform mixture, and compressing the mixture thus obtained to form tablets which may be optionally coated with a film coat or a sugar-coat.

In a most preferred embodiment of the invention, the present invention provides a process for preparing an IB-containing formulation comprising the steps of:

In the absence of moisture, fine particles of the NSAID, preferably IB, bicarbonate, preferably sodium bicarbonate, gelucire, preferably grade 44/14, and optionally other excipients are thoroughly mixed and converted into granules. Granules may be packaged as individual doses or may be compressed under low compression pressure to form tablets.

The mixing of the ingredients may be achieved in different ways. One way to mix the NSAID and bicarbonate and placed them in a fluidized bed system and while mixing, spray a solution of gelucire dissolved in a suitable vehicle preferably isopropanol onto the suspending dry mixture. Another method is to melt a mixture of gelucire and the NSAID at the lowest possible temperature and after drying of the mixture mix well with bicarbonate in the presence or absence of a suitable solvent preferably isopropranol.

A granulate composition of the present invention can be prepared by direct granulation of ibuprofen in the desired amount or, optionally, a first granulation of ibuprofen and one or more other additional ingredients as desired, or optionally, a second granulation after the first granulation with one or more additional ingredients.

The granulates obtained according to the above described methods are then screened, dried, combined with bicarbonate and any selected excipient(s) in the desired amounts and compressed in suitable molds for obtaining the desired tablets which can then be film coated, if desired.

In addition to good handling and workability, the tablets of the present invention provide complete dissolution of the active ingredient in about 10 minutes or less. Consequently the release is faster with respect to the commercially available ibuprofen based analgesic tablets (see example 2 below).

One skilled in the art readily recognizes that tablet compression provides certain benefits and characteristics in the administration and presentation of an active ingredient for adsorption. It is also known to those skilled in the art that the exact composition of a tablet partially dictates the method and attributes of the compression process. For example, it is generally known that too much compression may slow the release or disintegration of the tablet into smaller particles. It is therefore an embodiment of the invention to provide a tablet having been compressed within a range of compression values that promote or do not adversely affect disintegration of the tablet at the enhanced rate that forms an embodiment of the present invention.

It will be appreciated by the person skilled in the art that due to the different excipients used in the formulation and varying amounts thereof that for any compression pressure, different formulations will have different crushing strengths and disintegration times. Preferred dosage forms exhibit a crushing strength of 6.5-15 Kp and a disintegration time of less than 10 minutes at a compression force above 80 MPa. More preferred formulations exhibit a crushing strength of 6.5-15 Kp and a disintegration time of less than 10 minutes when compressed at a compression force in the range 100-140 MPa such as by a standard tabletting machine, e.g. a rotary tabletting machine. Such compression pressures include, 110 MPa, 120 MPa and 130 MPa. Especially preferred dosage forms exhibit a crushing strength of 6.5-15 Kp and a disintegration time of less than 10 minutes when compressed at all pressures in the range 100-140 MPa.

The disintegration time of the tablet formed in accordance with the present invention is less than 10 minutes as measured by the method described in the European Pharmacopoeia 1986, Ref V0.5.1.1 (updated 1995) (A. Disintegration Test for Tablets and Capsules). Preferred disintegration times are less than 6 minutes (e.g. 1-6 minutes), more preferably less than 5 minutes (e.g. 1-5 minutes) and most preferably 3 minutes or less (e.g. 1-3 minutes).

As used herein, a diluent or filler is used in its conventional pharmacological definition, and refers to an ingredient that adds necessary bulk to a formulation to prepare tablets of a desired size.

As used herein, a binder or adhesive is used in its conventional pharmacological definition, and refers to an ingredient that promotes the adhesion of the particles of the formulation.

As used herein, a disintegrator or disintegrating agent is used in its conventional pharmacological definition, and refers to an ingredient that promotes the post-administration break-up of the tablets into smaller particles for more ready drug availability.

As used herein, a lubricant or lubricating agent is used in its conventional pharmacological definition, and refers to an ingredient that enhances the flow of the tabletting material into the tablet dies, and prevents the tabletting material from sticking to punches and dies.

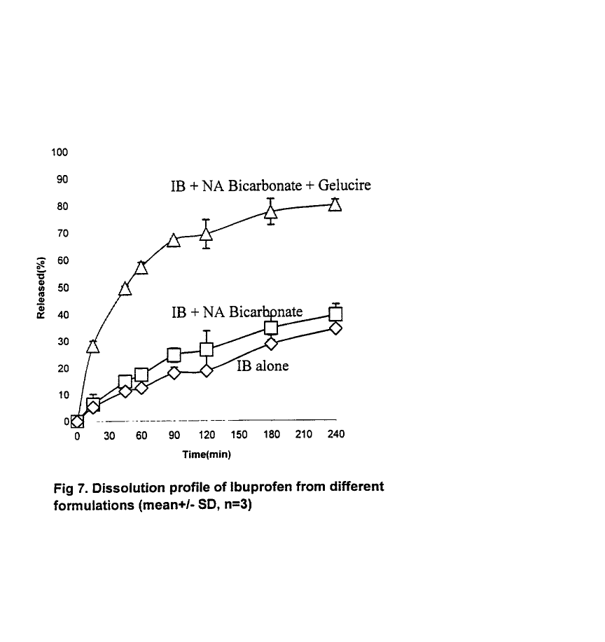

As used herein, enhanced absorption or similar terms and phrases relating to the relative speed, rate, and/or quantity of the bioavailability of the active agent. In accordance with the present invention, enhanced absorption is measured in reference to the standard in the industry, Motrin. In essence, the compositions of the present invention provide, to a patient in pain, a greater concentration of active agent faster, as compared to the bioavailability curve for Motrin. For example, see FIG. 7. In graphical or mathematical terms, enhanced absorption may be determined or quantified by using the area under the curve (AUC). As shown in FIG. 7, the extent and rate of absorption, as represented by the AUC, for the formulations of the present invention, delivers a greater amount of active agent in a shorter time frame as compared to Motrin. In accordance with the teachings of the present invention, it is important to determine enhanced absorption of a particular composition as it applies to a patient in pain, or data obtained from a patient or subject in pain.

In therapeutic use the dosage forms of the present invention are administered orally, thus the therapeutic dosage forms are presented in solid dosage form, preferably as a tablet. The dosage forms may be coated with a sugar or film coating, which dissolves substantially immediately the dosage form comes into contact with an aqueous medium. The composition may also be compressed onto a solid core of another material to form a solid formulation with an quick release outer coating. Alternatively, the compressed composition may be present in one or more layers of a multi-layer solid dosage form. In such formulations the remaining layers or core may comprise standard excipients to provide conventional, fast or slow release and are well within the knowledge of a person skilled in the art (e.g., see Remington's Pharmaceutical Sciences, 17th Edition, Ed Gennaro et al; or Ansel's “Introduction to Pharmaceutical Dosage Forms”, 2^{nd} edition, Henry Kimpton Publishers).

The following Examples illustrate specific formulations comprehended by the present invention, and methods for their preparation. The Examples are not intended to be limiting to the scope of the invention in any respect and should not be so construed.

### EXAMPLES

### Example 1

### Animal Model

Delayed absorption caused by vagal suppression that has previously been reported in the literature (e.g., Jamali & Axelson, 1997) was used to test the absorption rates of new ibuprofen formulations.

The animal models are Adult male Sprague-Dawley rats with body weight of 250-300 g, and which were cared for in accordance with the principles and guidelines of the Canadian Council of Animal Care. All rats were catheterized in the right jugular vein for sample collection.

An animal model having suppressed vagal properties were produced by administering (intraperitoneal injection) to the rats two 20 mg/kg doses of propantheline (test, n=6), an anticholinergic agent with known vagal suppressive properties, the first dose at 2 hours prior to administration of an NSAID, and the second at 1 hour prior.

One hour after the second dose of propantheline, 20 mg/kg doses of a commercially available ibuprofen tablet (Motrin 200 mg tablets, available from McNeil, Guelph, Canada, KIN 02186934, Batch 151979/(L)F316/Exp March 2001) were administered. The tablets were crushed gently and small pieces were administered into the stomach via a plastic tube followed by 0.5 mL tap water. Animals were fasted after the first dose of propantheline until 4 hours post-ibuprofen dose. They had free access to water.

Serial blood samples were withdrawn from the jugular vein cannula at suitable times post-ibuprofen dose. Plasma was separated and kept at −20° C. until analyzed for ibuprofen using a high performance chromatography method (Wright et al, 1992).

Results. Table 1 and FIG. 2 show that the absorption rate for ibuprofen in a vagally suppressed rat model was suppressed similar to what is reported in humans (Jamali & Kunz, 1999). Propantheline treatment (i.e., vagal suppression) caused a substantial and significant delay in absorption of ibuprofen. Notably, AUC(0-1), a reliable measure of absorption-rate was significantly reduced from 48.7 to 12.2 μg/h/mL.

TABLE 1
Bioavailability indices following oral administration of 20 mg/kg
of ibuprofen as crushed tablets to control and vagal-suppressed
(Pain Model) rats.

| | Tmax | Cmax | AUC (0-1) | AUC (0-8) |
|---|---|---|---|---|
| Rats | hour | μg/mL | μg/h/mL⁻¹ | μg/h/mL⁻¹ |
| Control | 0.28 | 40.4 | 48.7 | 139 |
| Pain Model | 0.75 | 13.8* | 12.2* | 81.8 |

*significantly different from Control (a = 0.05)

### Example 2

The rat model described in Example 1 was used to test whether an ibuprofen formulation can be made with rapid absorption-rate regardless of vagal suppression.

This example shows three formulations, a granule and two tablets, are rapidly absorbed even when vagal suppression is present.

Formulation 1 (ibuprofen granules): Ibuprofen 1000 g; sodium bicarbonate 497 g; and gelucire 41 g. To administer 20 mg/kg of ibuprofen to a 300 gram rat, 9.3 mg of this composition was dosed.

Formulation 2 (tablet, wet granulation): Ibuprofen 200 g, sodium bicarbonate 80 g, gelucire 15 g, hypromellose 20 g, pre-gelatanized starch 168.4 g; microcrystalline cellulose 84.0 g; sodium croscarmellose 28.0 g; and

magnesium stearate 3.0 g. Each tablet weighed 299 mg and contained 100 mg ibuprofen. To administer 20 mg/kg of ibuprofen to a 300 gram rat, the tablet was gently broken into small pieces and 17.9 mg of this composition was dosed.

Formulation 3 (tablet, dry granulation): Ibuprofen granule 583.7 g (Ibuprofen 200 g, Sodium bicarbonate 80 g, Gelucire 15 g, Maize starch 17.7 g, Sodium croscarmellose 42.0 g, microcrystalline cellulose 58.3.0 g, and precipitated silica 11.7); pre-gelatanized starch 361.5 g, microcrystalline cellulose 180.8 g, Sodium croscarmellose 41.0 g, and magnesium stearate 6.0 g. Each tablet weighed 586.5 mg and contained 100 mg ibuprofen. To administer 20 mg/kg of ibuprofen to a 300 gram rat, the tablet was gently broken into small pieces and 35.2 mg of this composition was dosed.

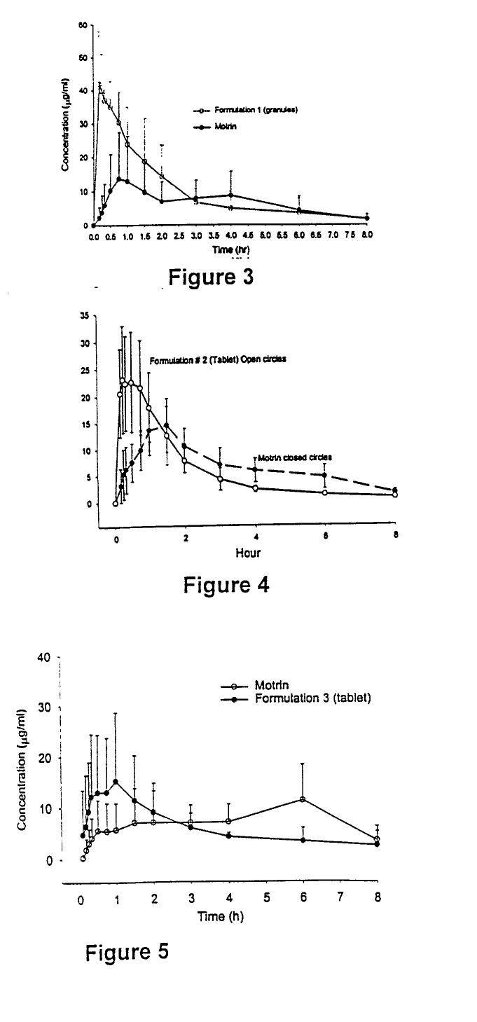

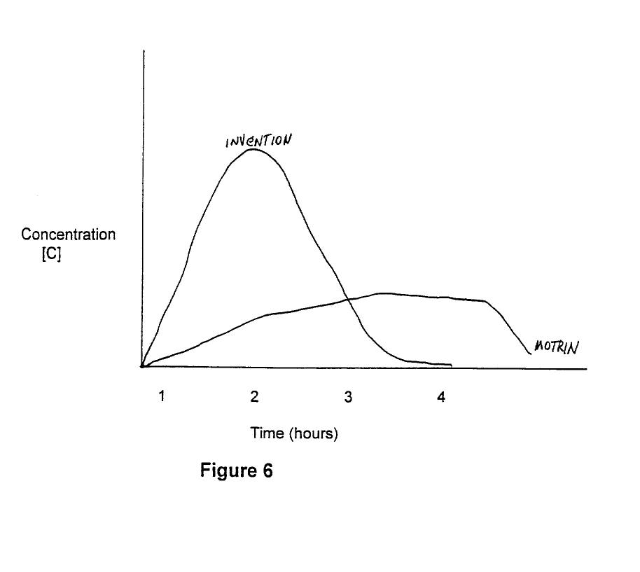

In the vagal-suppressed rat, all of the invented formulations exhibited significantly more rapid absorption than Motrin (20 mg/kg of ibuprofen as crushed Motrin tablets). See Tables 2-4 and FIGS. 3-5.

TABLE 2
(Formulation #1)

| | Tmax | Cmax | AUC (0-1) | AUC (0-8) |
|---|---|---|---|---|
| Formulation | h | μg/mL | μg/h/mL⁻¹ | μg/h/mL⁻¹ |
| Motrin | 0.75 | 13.8 | 12.2 | 81.8 |
| Formulation #1 | 0.17* | 42.0* | 45.6* | 123 |

*Significantly (a = 0.05) different from Motrin

Formulation #1 granules (Table 2) exhibited the fastest absorption-rate. The first collected sample (10 minutes post-dose) contained the highest ibuprofen concentration. The plasma ibuprofen concentration-time curve had a smooth pattern with no evidence of multi-peaking. See FIG. 3.

As expected and is shown in FIG. 2, the plasma ibuprofen concentration-time curve following Motrin administration to vagal-suppressed rats demonstrated a slower and erratic absorption than Formulation #1 and also Motrin in control animals (see FIG. 3).

TABLE 3
(Formulation #2)

| | Tmax | Cmax | AUC (0-1) | AUC (0-8) |
|---|---|---|---|---|
| Formulation | h | μg/mL | μg/h/mL⁻¹ | μg/h/mL⁻¹ |
| Motrin | 1.5 | 14.5 | 10.4 | 81.2 |
| Formulation #2 | 0.25* | 19.7 | 24.7* | 63.1 |

*Significantly (a = 0.05) different from Motrin

TABLE 4
(Formulation #3)

| | Tmax | Cmax | AUC (0-1) | AUC (0-8) |
|---|---|---|---|---|
| Formulation | h | μg/mL | μg/h/mL⁻¹ | μg/h/mL⁻¹ |
| Motrin | 6.0 | 7.12 | 6.12 | 88.8 |
| Formulation #3 | 0.5* | 13.0 | 16.2* | 75.8 |

*Significantly (a = 0.05) different from Motrin

Both tablet formulations exhibited significantly more rapid absorption than Motrin as reflected by over two fold increase in AUC(0-1) for both Formulation #2 (FIG. 4, Table 3) and Formulation #3 (FIG. 5, Table 4).

### Conclusions

1. Absorption profile of ibuprofen in vagal-suppressed (propantheline-treated) rats is similar to that of humans following dental surgery.

2. Absorption of a commercially available ibuprofen tablet is slowed down in both propantheline-treated rats and humans following dental surgery

3. Ibuprofen granules prepared under conditions described here have significantly improved absorption rate in propantheline-treated rats as compared with a crushed commercially available ibuprofen tablet.

4. Ibuprofen tablets prepared under conditions described here have significantly improved absorption rate in propantheline-treated rats as compared with a crushed commercially available ibuprofen tablet.

### Example 3

### In Vitro Dissolution Test

Using the U.S. Pharmacoipoeia Apparatus II, the dissolution rates of ibuprofen alone, ibuprofen plus sodium bicarbonate (1:1 molar based), and ibuprofen plus sodium bicarbonate (1:1 molar based) plus gelucire (5% total weight) were assessed. The apparatus contained 2 g of NaCl and 7 mL of concentrated HCI (pH 1.2) in 900 mL water. The medium was kept at 37° C., and was stirred with a rotating paddle at 75 rounds per minute. Ibuprofen was detected at 232 nm. The amount dissolved per unit time is shown in FIG. 7.

Although the present invention has been described in terms of a particular preferred embodiments, it is not limited to those embodiments. Alternative embodiments, examples, and modifications which would still be encompassed by the invention may be made by those skilled in the art, particularly in light of the foregoing teachings.

1. A pharmaceutical composition comprising a non-steroidal anti-inflammatory active agent, at least one disintegration agent, and at least one anti-precipitation agent.

2. The pharmaceutical composition of claim 1 wherein the anti-inflammatory active agent is selected from the group consisting of piroxicam, meloxicam, indomethacin, fenoprofen, keterolac, naproxen, and ibuprofen.

3. The pharmaceutical composition of claim 1 wherein the disintegration agent is an alkali metal carbonate.

4. The pharmaceutical composition of claim 4 wherein the alkali metal carbonate is sodium bicarbonate.

5. The pharmaceutical composition of claim 1 wherein the anti-precipitation agent is a fatty acid ester.

6. The pharmaceutical agent of claim 5 wherein the fatty acid ester is gelucire.

7. The pharmaceutical composition of claim 1 comprising ibuprofen, sodium bicarbonate, and gelucire.

8. The pharmaceutical composition of claim 7 further comprising hypromellose, pre-gelatinized starch, microcrystalline cellulose, sodium croscarmellose, and magnesium stearate.

9. The pharmaceutical composition of claim 8 further comprising maize starch, and precipitated silica.

10. A method for the treatment of inflammation comprising supplying an anti-inflammation formulation, said formulation comprising a non-steroidal anti-inflammatory active agent and a disintegrating agent comprising an alkali metal carbonate; and administering said formulation.

11. The method of claim 10 further comprising supplying an anti-inflammation formulation further comprising an anti-precipitation agent.

12. The method of claim 11 wherein the anti-inflammation formulation comprises ibuprofen, a bicarbonate, and a fatty acid ester.
